(19) United States
(12) Patent Application Publication
(10) Pub. No.: US 2003/0224020 A1
(43) Pub. Date: Dec. 4, 2003

(54) Pharmaceutical preparation of botulinum neurotoxin, methods of synthesis and methods of clinical use

(76) Inventors: Alexander F. Zabudkin, Donetsk (UA); Juri M. Krasnopolsky, Charkov (UA); Aleksandr M. Itkin, San Diego, CA (US); Dmitry M. Itkin, San Diego, CA (US)

Correspondence Address: Law Offices of James K. Sakaguchi, 3496 Paseo Ancho, Carlsbad, CA, 92009, US

(21) Appl. No.: 10/447,417

(22) Filed: May 28, 2003

Related U.S. Application Data
(60) Provisional application No. 60/385,286, filed on May 31, 2002.

(51) Int. Cl. A61K 39/08

(52) U.S. Cl. 424/239.1

(57) ABSTRACT

A pharmaceutical preparation of botulinum neurotoxins free of human blood products (such as human albumin), the preparation comprising a botulinum neurotoxin incorporated in phosphatidylcholine liposomes.

The benefit of U.S. Provisional Application No. 60/385,286, filed May 31, 2002, under 35 U.S.C. Section 119(e), and any other applicable laws, is hereby claimed.

### FIELD OF THE INVENTION

The present invention relates generally to pharmaceutical compositions. In particular, the present invention relates to novel pharmaceutical preparations containing botulinum neurotoxin and a method for making the pharmaceutical preparations.

### BACKGROUND OF THE INVENTION

The present invention relates to a novel pharmacological composition (dosage form) of botulinum toxins.

Botulinum Neurotoxins

All types of botulinum neurotoxin (botulinum toxin), including A, B, C₁, C₂, D, E, F and G are produced by strains of Clostridium botulinum bacterium (“C. botulinum”). C. botulinum is the anaerobic, gram positive rod implicated in producing the most serious form of food poisoning known as botulism. This condition is induced by the botulinum neurotoxins which are preformed by the bacterium in foods under anaerobic conditions. When injested, the botulinum neurotoxin is absorbed through the digestive tract and delivered to the site of action, the neuromuscular synaptic junction, via the vascular circulatory system. The strains producing botulinum toxin type A are the predominant cause of botulism in the United States. (Simpson, L L. Pharmacol Review 1981;(33):155-188).

Current clinical use of commercially available preparations of botulinum toxins (type A toxins include BOTOX®, BOTOX Cosmetic®, Dysport™, and type B toxins include MYOBLOC® and NEUROBLOC®) include achalasia, anal fissure, anismus, blepharospasm, cerebral palsy, cervical dystonia, cervicogenic headache, hemifacial spasm, dyshidrotic eczema, dysphagia, dysphonia, esophageal dysmotility, esophageal muscular ring, esotropia (infantile), eyelift, facial myokemia, gait disturbances (idiopathic toe-walking), generalized dystonia, hemifacial spasm, hyperfunctional facial lines (glabellar, forehead, crow feet, downturned angles of the mouth), hyperhidrosis, incontinence (spinal cord injury), migraine headache, myoclonus, myofascial pain syndrome, obstructive urinary symptoms, pancreas divisum pancreatitis, Parkinson's disease, puborectalis syndrome, reduction of surgical scar tension, salivary hypersecretion, sialocele, sixth nerve palsy, spasticity, speech/voice disorders, strabismus, surgery adjunct (ophthalmic), tardive dyskinesia, temporomandibular joint disorders, tension headache, thoracic outlet syndrome, torsion dystonia, torticolis, Tourette's syndrome, tremor, whiplash-associated neck pain (Botulinum toxin type A, Botulinum toxin type B; in MICROMEDEX® Healthcare Series, Drugdex Drug Evaluation; 2003).

The botulinum toxin molecules of all serotypes (A through G) are synthesized as a single polypeptide chain with a molecular mass of approximately 150 kD. The protein is then cleaved by a bacterial protease, and an activated protein is formed comprised of two peptide chains, a light chain (50 kD) and heavy chain (100 kD) attached to each other by a disulfide bond. The toxin mediated muscle paralysis is achieved by internalization via botulinum toxin receptor, reduction of disulfide bond in the endosome and translocation of heavy chain inside the motor axon terminal, and irreversible inhibition of acetylcholine release from the axon terminal membrane. Although the mechanism of action appears similar for all types of botulinum toxin, each serotype inhibits acetylcholine release via action at different cellular targets (Brin, Me. Muscle and Nerve 1997; Supplement 6:S146-S168):

TABLE 1
Protein targets of botulinum toxin (Adapted from Brin ME,
referenced above).

| TOXIN TYPE | TARGET SUBSTRATE | CELL |
|---|---|---|
| A | SNAP-25 | Neuron |
| B | VAMP/Synaptobrevin | Neuron |
| C | Syntax 1A, 1B. | Neuron |
| | SNAP-25 | Neuron |
| D | VAMP/Synaptobrevin | Neuron |
| | Cellubrevin | All cells |
| E | SNAP-25 | Neurin |
| F | VAMP/Synaptobrevin | Neuron |
| | Cellubrevin | All cells |
| G | VAMP/Synaptobrevin | Neuron |

The first successful purification of type A botulinum neurotoxin was accomplished by Snipe and Sommer at the Hooper Foundation at the University of California in 1928 when 90% of the crude toxin could be precipitated from the spent culture at pH 3.5. The clinical application of botulinum toxin required a more rigorous purification process while minimizing the inactivated toxoid byproduct. Such a crystalline form of botulinum toxin type A was prepared by Schantz E J, Food Research Institute, Department of Food Microbiology and Toxicology at the University of Wisconsin-Madison. (Schantz E J, Johnson E A. Microbiol Reviews 1992;56(1):80-99). This is the form that was the biologic substance approved by the U.S. Food and Drug Administration (FDA) as an injectable substance for clinical use. This is the form currently used in BOTOX® and BOTOX Cosmetic® (Allergan, Inc., Irvine, Calif.).

All of the serotypes of botulinum neurotoxins are released by C. botulinum as complexes of the particular botulinum neurotoxin protein molecule along with associated non-toxin proteins. For clarity, as used herein, the words or phrases have the definitions set forth below.

“Botulinum neurotoxin” or “botulinum toxin” means the botulinum neurotoxin protein molecule either (i) in association with one or more of the associated proteins of the complex or (ii) as isolated from the associated proteins of the complex.

“Botulinum neurotoxin complex” or “botulinum toxin complex” means the botulinum toxin protein molecule along with its associated non-toxin proteins.

“Purified botulinum neurotoxin” or “purified botulinum toxin” means the botulinum neurotoxin protein molecule dissociated from the non-toxin proteins of the complex.

Type A botulinum toxin is a part of a complex consisting of the 150 kD dipeptide toxin and a group of non-covalently bound proteins. These proteins do not possess toxic properties, but serve as natural stabilizers for the neurotoxin itself. The non-neurotoxin proteins contained in type A botulinum toxin exhibit hemaglutinin properties. All botulinum toxin complexes can be subdivided according to various molecular sizes into M (medium), L (large) and LL (very large):

TABLE 2
Molecular sizes of various botulinum neurotoxin complexes (Adapted
from Ohishi I, Sakaguchi G. Apl. Inviron Microbiol 1974;
28: 923-928; and Kozaki S et al. Jpn
J Med Sci Biol 1974; 28: 70-72).

| | | | Sedimentation |
|---|---|---|---|
| Size type | Toxin type | Molecular Weight, kD | coefficient |
| LL | A | 900 | 19S |
| L | A, B, C, G | 450-500 | 16S |
| M | A, B, C₁, D, E, F, G | 235-350 | 10-12S |

BOTOX® and BOTOX Cosmetic® (Allergan, Inc, Irvine, Calif.), the two identical preparations currently approved by the U.S. FDA for certain uses, contain LL type A botulinum neurotoxin having a 900 kD molecular weight. This particular complex was found to have the highest muscle weakening efficacy which was comparable to that of pure type A neurotoxin (150 kD) (Aoki KR, Europ J Neurol 1999;6(suppl 4):S3-S10).

The toxicity of botulinum neurotoxins are commonly measured in Mouse Units (MU), defined as MLD₅₀ (Mouse Lethal Dose 50%). MLD50 is defined as the dose of botulinum toxin introduced intraperitoneally in white female mice weighing 18-22 g resulting in 50% lethality. Schantz E J and Kautter D A described such a standardized assay for Clostridium botulinum toxins. (Schantz E J and Kautter D A in J of AOAC; 1978;61:96-99).

The pharmaceutical-grade type A botulinum toxin complex should have (1) specific toxicity of 3×10⁷ MLD₅₀ (±20%) per mg; (2) a maximum absorbance at 278 nm when dissolved in 0.05 M sodium phosphate buffer at pH 6.8; (3) an A₂₆₀/A₂₇₈ ratio of 0.6 or less; (4) an extinction coefficient (absorbancy) of 1.65 for 1 mg of toxin complex per 1 ml in a 1-cm light path. (Schantz E J, Johnson E A. Microbiol Reviews 1992;56(1):80-99). The purified type A neurotoxin chromatographically separated from the non-neurotoxic peptides should have a specific toxicity of 9×10⁷ to 1×10⁸ MLD₅₀ per mg (DasGupta BR, Sugiyama H, in Perspectives in Toxicology 1977, p87 (Bernheimer A W, Ed.,) New York: John Wiley), and an extinction coefficient (absorbancy) of 1.63 for 1 mg of pure neurotoxin per 1 ml in a 1-cm light path (DasGupta B R, Sathyamoorthy V. Toxicon 1984;22(3):415-424).

It is a well-known phenomenon, that botulinum neurotoxins injected for treatment of hyperactive muscles may induce the development of neutralizing antibodies, making further treatments less efficacious. This phenomenon involves recognition of the botulinum neurotoxin proteins as foreign antigens by the human or mammalian cellular immune system and a consequent production of the antibodies. A former preparation of BOTOX® (the original batch approved by the U.S. FDA in 1979) was shown to be more immunogenic than the new BOTOX® (the new batch approved by the U.S. FDA in 1997) (Aoki K R Eur J Neurol 1999;6 (suppl 4):S3-S 10). Several explanations may be postulated for the increased immunogenicity of different batches of the type A botulinum toxin. First of all, clinicians were using high doses of the type A botulinum toxin (in excess of 600 U). It has been shown that use of less than 300 U per treatment substantially decreases production of neutralizing antibodies. Secondly, the old batch of BOTOX® possibly contained inactivated botulinum toxoid, which increased immunogenicity of the preparation. Finding an alternative composition of the type A botulinum neurotoxin complex or pure type A botulinum neurotoxin may decrease induction of the neutralizing antibodies.

There is one major limitation in all currently commercially available preparations of botulinum toxins (namely, BOTOX®), BOTOX Cosmetix®, DYSPORT®, and MYOBLOC™) and newer patented preparations (for example, U.S. Pat. Nos. 5,512,547; 5,696,077; 5,756,468; 6,312,708; 6,444,209). All these preparations contain human albumin serving as the excipient substance which serves to stabilize the botulinum neurotoxin complex or pure botulinum neurotoxin in solution and during the lyophilization process. In fact, it has been shown that presence of bovine or human albumin allows ≧90% recovery of original toxicity after lyophilization, making albumin an attractive and readily available excipient stabilizer (Goodnough M C, Johnson E A. App Envir Microbiol 1992;58(10):3426-3428).

The need for a stabilizing excipient stems from the fact that botulinum neurotoxins are very susceptible to denaturation (surface denaturation, heat lability, alkaline conditions lability, etc). The current commercially available preparation of the type A botulinum neurotoxin (BOTOX® and BOTOX Cosmetic®) owes its stability to human albumin excipient. Lyophilization (freeze-drying method), a widely utilized, inexpensive, and practically acceptable method of drug preservation in the pharmaceutical industry, is the preferred method of preparation of current commercial and investigational types of botulinum toxins and their pharmacologic complexes. All of the lyophilized preparations contain human albumin as a stabilizing excipient. Human albumin for injections is prepared from pulled donor blood. This blood undergoes vigorous screening for bloodborn pathogenic organisms such as hepatitis B, hepatitis C, and human immunodeficiency viruses. Although there have been no reports of human albumin serving as a vector for transmission of bloodborn diseases, such transmission cannot be statistically excluded. One way to eliminate the chance of transmitting bloodborn pathogens, such as hepatitis B, hepatitis C, or human immunodeficiency virus, is to create a preparation that contains no human blood products (i.e. human albumin) but would remain stable during the lyophilization process, during shelf storage, and after reconstitution for injection.

A published International Application, WO 01/58472 A2, discloses an attempt to formulate a pharmaceutical composition containing botulinum toxin which is free of human albumin. This published application describes a composition comprising botulinum toxin, sodium chloride and a polysaccharide as a stabilizing agent. However, there still exists a need for a more stable and effective preparation for the delivery of botulinum toxin.

Liposomes

Liposomes may serve as a drug delivery vehicle. The utility of liposomes, and related prior art, have been eloquently described in U.S. Pat. No. 5,409,698, issued in 1995 to Anderson P M et al.

In short, liposomes are closed membrane systems that are formed spontaneously in a dispersion of phospholipids in water. The structure is made of one or several concentric bilayers formed by phospholipid molecules in such a manner that subdivides the system into hydrophilic and hydrophobic compartments. During liposomal drug preparation, water and hydrophilic drug solution gets spontaneously incorporated in the hydrophilic compartment of the liposome, while hydrophobic substances get incorporated within the hydrophobic compartment of the phospholipids bilayer. Various substances can be incorporated into liposomes. These include commercially available antibiotics, antifungal medications, cytokines, vaccines, immunosuppressants, and chemotherapeutic agents, as well as research compounds incorporating various proteins and other bioactive compounds inside the liposomes. Reduction of major organ toxicity was noted for all liposomal compounds.

Liposomes can be prepared by multiple methods. The most commonly used method is the film method, whereby lipids are dissolved in an organic solvent with added hydrophobic biolactive compound and then dried in a round-bottomed flask. Appropriate aliquots of water with dissolved hydrophilic bioactive compound are then added, and the flask is agitated by a gentle swirling. Substances aiding in the emulsification process (such as lactose) are commonly added to aid the emulsification process. The liposomes spontaneously form as multilamellar vesicles (MLVs). However, the small unilamellar vesicles (SUVs) are considered to be more desirable drug delivery vehicles because of their increased longevity in vivo. Preparation of SUVs requires treatment of the lipid in water emulsion by various methods (such as ultrasonic waves, pressure-assisted filtration through microporous filter, etc.) with the consequent step-wise filtration through smaller and smaller porous filters to obtain the 0.25-0.35 mkm liposomes, in other words, the SUVs.

Because the liposomal membrane consists of natural phospholipids, liposomes possess numerous attractive properties. Liposomes are non-immunogenic, non-toxic and bio-degradable. Tissue cells can absorb liposomes by incorporating them into the cell membrane allowing for a direct delivery of the incorporated medication to the target cells. Liposomes act as a protective environment for the incorporated medication thereby limiting desiccation and degradation by tissue enzymes. Liposomes increase stability of the incorporated proteins maintaining their native tertiary structure and biological activity. Although liposomes may serve as an immunogenic adjuvant in liposomal vaccine preparations, their adjuvant properties are not more than those of human albumin, therefore, decreasing concern of formation of neutralizing antibodies. (see all references mentioned in U.S. Pat. No. 5,409,698).

U.S. Pat. No. 6,312,708, issued to Donovan, S. (Allergan Sales, Inc; Irvine, Calif.), describes a botulinum toxin implant having polymeric biocompatible, biodegradable microspheres capable of controlled, pulsatile, sustained release of all types of botulinum neurotoxins. U.S. Pat. No. 6,312,708 does not mention or suggest phospholipids of any kind as being a suitable biodegradable carrier for polymeric microspheres. Moreover, the botulinum toxin implants described in U.S. Pat. No. 6,312,708 incorporate the known, commercially available preparations of botulinum toxins (such as BOTOX® or BOTOX Cosmetic®, all of which contain human albumin as a stabilizing excipient.

All references cited within this herein are hereby incorporated by reference herein in their entireties.

### SUMMARY OF THE INVENTION

The present invention provides a pharmaceutical preparation comprising a formulation of phosphatidylcholine liposomes, lactose, sodium chloride and type A botulinum neurotoxin complex or purified type A botulinum neurotoxin. We have discovered that pharmaceutical preparations made of a lyophilized formulation of phosphatidylcholine liposomes, lactose, sodium chloride and type A botulinum neurotoxin complex or purified type A botulinum neurotoxin is a stable pharmacological preparation that:

(1) contains no human blood products (such as human albumin), thereby decreasing the chance of transmission of bloodborn infections such as hepatitis B, hepatitis C, and human immunodeficiency virus;

(2) allows for >75% recovery of the toxicity following the lyophilization process, and more preferably >90% of the toxicity following the lyophilization process; and

(3) has substantially no loss of neurotoxin potency for a period of one year, and more preferably four years, when stored at −5 to 37 degrees C.; and

(4) has decreased immunoadjuvant properties, thereby decreasing the chance of neutralizing antibody formation after injection of the preparation into animal models or patients.

We also have discovered that the new liposomal preparation of type A botulinum neurotoxin complex or purified type A botulinum neurotoxin can be used for selective, partial, temporary chemical denervation of the clinically relevant muscle groups in mammals and humans in a manner similar to the currently commercially available preparations of type A botulinum neurotoxin.

We have discovered that pharmaceutical preparations made of a lyophilized formulation of phosphatidylcholine liposomes, lactose, sodium chloride, and any of the other types of botulinum neurotoxin complexes (types B, C1, C2, D, E, F or G) or purified botulinum neurotoxins (types B, C1, C2, D, E, F or G) are stable pharmaceutical preparations that:

(1) contain no human blood products (such as human albumin), thereby decreasing a chance of transmission of bloodborn infections such as hepatitis B, hepaptitis C, and human immunodeficiency viruses;

(2) allow for >75% recovery of the toxicity following lyophilization process, and more preferably >90% of the toxicity following the lyophilization process;

(3) has substantially no loss of neurotoxin potency for a period of one year, and more preferably four years, when stored at −5 to 37 degrees C.; and

(4) have decreased immunoadjuvant properties, thereby decreasing the chance of neutralizing antibody formation after injection of the preparation into animal models or patients.

We also have discovered that the new liposomal preparations of types B, C1, C2, D, E, F, or G botulinum neurotoxin complexes or their corresponding purified toxins can be used for selective, partial, temporary chemical denervation of the clinically relevant muscle groups in mammals and humans in a similar manner to the currently commercially available preparations of type A botulinum neurotoxin.

### DETAILED DESCRIPTION OF THE INVENTION

The preferred pharmaceutical compositions of the present invention have the following composition:

| | | |
|---|---|---|
| Botulinum type A neurotoxin complex (95-98% purity) | 100 | MU |
| Phosphatidylcholine (lethitin), egg-derived | 0.01 | mg |
| Lactose | 2.5 | mg |
| Alternatively: | | |
| Botulinum type A neurotoxin complex (95-98% purity) | 100 | MU |
| Phosphatidylcholine (lethitin), egg-derived | 0.01 | mg |
| Lactose | 5 | mg |
| Alternatively: | | |
| Botulinum type A neurotoxin complex (95-98% purity) | 100 | MU |
| Phosphatidylcholine (lethitin), egg-derived | 0.01 | mg |
| Alternatively: | | |
| Purified botulinum type A neurotoxin (95-98% purity) | 100 | MU |
| Phosphatidylcholine (lethitin), egg-derived | 0.01 | mg |
| Lactose | 2.5 | mg |
| Alternatively: | | |
| Purified botulinum type A neurotoxin (95-98% purity) | 100 | MU |
| Phosphatidylcholine (lethitin), egg-derived | 0.01 | mg |
| Lactose | 5 | mg |
| Alternatively: | | |
| Purified botulinum type A neurotoxin (95-98% purity) | 100 | MU |
| Phosphatidylcholine (lethitin), egg-derived | 0.01 | mg |

The botulinum type A neurotoxin complex and purified neurotoxin are produced from the C. botulinum type A 189 strain. The process of the toxin production involves culture incubation in static, anaerobic conditions, volume 5-10 liters, in a culture medium of the following composition:

| 1. Nitrate | 120-150 mg % |
|---|---|
| 2. Peptone | 2.0-2.3% |
| 3. Triptophan | trace |
| 4. Cysteine | 100-140 mg % |
| 5. Sodium chloride | 0.85-0.92% |
| 6. Iron | 0.05-0.2 mg % |
| 7. pH | 6.7-6.9 |

Purification of the type A botulinum neurotoxin complex is done as previously described by Schantz E J, Johnson EA. Microbiol Reviews 1992;56(1):80-99. Likewise, purification of the type A botulinum neurotoxin is done as previously described by DasGupta BR, Sathyamoorthy V. Toxicon 1984;22(3):415-424.

### EXAMPLES

The following examples illustrate the methods and means of production of the liposomal combinations of botulinum type A neurotoxin complex and purified botulinum type A neurotoxin, according to the present invention.

### Example 1

Initially, a flask is filled with a solution of phosphatidylcholine in ethanol containing 0.1 gram of lipid. The solution is subjected to evaporation in a rotating evaporator at a temperature of 30-35 degrees C. until a lipid film is formed. After the evaporation process is completed, an inert gas is passed through the flask of solution for 5 minutes. The lipid film is then re-suspended in 10 liters of sterile 0.9% sodium chloride solution with phosphate buffer (pH 7.0-7.4) containing 1 mg of botulinum type A neurotoxin complex (95-98% purity). After the lipid film is successfully re-suspended from the flask walls, the resulting emulsion is thoroughly mixed for 30 minutes until homogeneous emulsion is produced. Such emulsion is then transferred into a homogenizing reactor and the emulsion is homogenized at a pressure of 60 MPa and a temperature of 30-35 degrees C. The homogenization process is controlled by monitoring optical density values in the vial at a wavelength of 540 nm with a light path thickness of 3 mm. When an optical density of 0.1-0.12 is achieved, 25 grams of lactose is added to the emulsion. The resulting emulsion is then sequentially filtered (for example, in a Millipore, Inc. filtering device), initially through a 0.65 micron filter, then through a 0.45 micron filter, and finally through a 0.22 micron filter. The resulting sterile emulsion is then distributed into vials or ampoules, each containing 0.1 ml of sterile emulsion. The vials or ampoules are deep frozen at a temperature of −70 degrees C. for 48 hours, followed by lyophilization (deep-freeze drying). After lyophilization, the vials are hermetically sealed with an atmosphere of inert gas introduced over the lypophilized emulsion in the vial.

### Example 2

Initially, a flask is filled with a solution of phosphatidylcholine in ethanol containing 0.1 gram of lipid. The solution is subjected to evaporation in a rotating evaporator at a temperature of 30-35 degrees C. until a lipid film is formed. After the evaporation process is completed, an inert gas is passed through the flask of solution for 5 minutes. The lipid film is then re-suspended in 10 liters of sterile 0.9% sodium chloride solution with phosphate buffer (pH 7.0-7.4) containing 0.1 mg of botulinum type A neurotoxin complex (95-98% purity). After the lipid film is successfully re-suspended from the flask walls, the resulting emulsion is thoroughly mixed for 30 minutes until homogeneous emulsion is produced. Such emulsion is then transferred into a homogenizing reactor and the emulsion is homogenized at a pressure of 60 MPa and a temperature of 30-35 degrees C. The homogenization process is controlled by monitoring optical density values in the vial at a wavelength of 540 nm with a light path thickness of 3 mm. When an optical density of 0.1-0.12 is achieved, 5 grams of lactose is added to the emulsion. The resulting emulsion is then sequentially filtered (for example, in a Millipore, Inc. filtering device), initially through a 0.65 micron filter, then through a 0.45 micron filter, and finally through a 0.22 micron filter. The resulting sterile emulsion is then distributed into vials or ampoules, each containing 0.1 ml of sterile emulsion. The vials or ampoules are deep frozen at a temperature of −70 degrees C. for 48 hours, followed by lyophilization (deep-freeze drying). After lyophilization, the vials are hermetically sealed with an atmosphere of inert gas introduced over the lypophilized emulsion in the vial.

### Example 3

Initially, a flask is filled with a solution of phosphatidylcholine in ethanol containing 0.1 gram of lipid. The solution is subjected to evaporation in a rotating evaporator at a temperature of 30-35 degrees C. until a lipid film is formed. After the evaporation process is completed, an inert gas is passed through the flask of solution for 5 minutes. The lipid film is then re-suspended in 10 liters of sterile 0.9% sodium chloride solution with phosphate buffer (pH 7.0-7.4) containing 0.1 mg of botulinum type A neurotoxin complex (95-98% purity). After the lipid film is successfully re-suspended from the flask walls, the resulting emulsion is thoroughly mixed for 30 minutes until homogeneous emulsion is produced. Such emulsion is then transferred into a homogenizing reactor and the emulsion is homogenized at a pressure of 60 MPa and a temperature of 30-35 degrees C. The homogenization process is controlled by monitoring optical density values in the vial at a wavelength of 540 nm with a light path thickness of 3 mm. The resulting emulsion is then sequentially filtered (for example, in a Millipore, Inc. filtering device), initially through a 0.65 micron filter, then through a 0.45 micron filter, and finally through a 0.22 micron filter. The resulting sterile emulsion is then distributed into vials or ampoules, each containing 0.1 ml of sterile emulsion. The vials or ampoules are deep frozen at a temperature of −70 degrees C. for 48 hours, followed by lyophilization (deep-freeze drying). After lyophilization, the vials are hermetically sealed with an atmosphere of inert gas introduced over the lypophilized emulsion in the vial.

### Example 4

Initially, a flask is filled with a solution of phosphatidylcholine in ethanol containing 0.1 gram of lipid. The solution is subjected to evaporation in a rotating evaporator at a temperature of 30-35 degrees C. until a lipid film is formed. After the evaporation process is completed, an inert gas is passed through the flask of solution for 5 minutes. The lipid film is then re-suspended in 10 liters of sterile 0.9% sodium chloride solution with phosphate buffer (pH 7.0-7.4) containing 1 mg of botulinum type A neurotoxin complex (95-98% purity). After the lipid film is successfully re-suspended from the flask walls, the resulting emulsion is thoroughly mixed for 30 minutes until homogeneous emulsion is produced. Such emulsion is then transferred into a homogenizing reactor and the emulsion is homogenized at a pressure of 60 MPa and a temperature of 30-35 degrees C. The homogenization process is controlled by monitoring optical density values in the vial at a wavelength of 540 nm with a light path thickness of 3 mm. The resulting emulsion is then sequentially filtered (for example, in a Millipore, Inc. filtering device), initially through a 0.65 micron filter, then through a 0.45 micron filter, and finally through a 0.22 micron filter. The resulting sterile emulsion is then distributed into vials or ampoules, each containing 0.1 ml of sterile emulsion. The vials or ampoules are deep frozen at a temperature of −70 degrees C. for 48 hours, followed by lyophilization (deep-freeze drying). After lyophilization, the vials are hermetically sealed with an atmosphere of inert gas introduced over the lypophilized emulsion in the vial.

The following table contains physical and chemical characteristics of the products described in Examples 1 through 4. The characteristics were defined in the experiments using a solution prepared from the contents of the lyophilized vials reconstituted in sterile injectable normal saline.

TABLE 3
Physical and Chemical Characteristics of the
Preparations of Examples 1-4.

| | Examples | | | |
|---|---|---|---|---|
| Characteristics | 1 | 2 | 3 | 4 |
| Liposome size, nm | 240 ± 15 | 300 ± 40 | 220 ± 10 | 380 ± 40 |
| | 7.10 | 7.25 | 7.20 | 7.20 |
| Residual water | 1.10 | 3.00 | 1.22 | 3.81 |
| content, % | | | | |
| Sterility | Sterile | Sterile | Sterile | Sterile |
| Lipid content in 1 ml, | 0.0097 | 0.0091 | 0.00098 | 0.00094 |
| mg | | | | |

### Example 5

Initially, a flask is filled with a solution of phosphatidylcholine in ethanol containing 0.1 gram of lipid. The solution is subjected to evaporation in a rotating evaporator at a temperature of 30-35 degrees C. until a lipid film is formed. After the evaporation process is completed, an inert gas is passed through the flask of solution for 5 minutes. The lipid film is then re-suspended in 10 liters of sterile 0.9% sodium chloride solution with phosphate buffer (pH 7.0-7.4) containing 1 mg of purified botulinum type A neurotoxin complex (95-98% purity). After the lipid film is successfully re-suspended from the flask walls, the resulting emulsion is thoroughly mixed for 30 minutes until homogeneous emulsion is produced. Such emulsion is then transferred into a homogenizing reactor and the emulsion is homogenized at a pressure of 60 MPa and a temperature of 30-35 degrees C. The homogenization process is controlled by monitoring optical density values in the vial at a wavelength of 540 nm with a light path thickness of 3 mm. When an optical density of 0.1-0.12 is achieved, 25 grams of lactose is added to the emulsion. The resulting emulsion is then sequentially filtered (for example, in a Millipore, Inc. filtering device), initially through a 0.65 micron filter, then through a 0.45 micron filter, and finally through a 0.22 micron filter. The resulting sterile emulsion is then distributed into vials or ampoules, each containing 0.1 ml of sterile emulsion. The vials or ampoules are deep frozen at a temperature of −70 degrees C. for 48 hours, followed by lyophilization (deep-freeze drying). After lyophilization, the vials are hermetically sealed with an atmosphere of inert gas introduced over the lypophilized emulsion in the vial.

### Example 6

Initially, a flask is filled with a solution of phosphatidylcholine in ethanol containing 0.1 gram of lipid. The solution is subjected to evaporation in a rotating evaporator at a temperature of 30-35 degrees C. until a lipid film is formed. After the evaporation process is completed, an inert gas is passed through the flask of solution for 5 minutes. The lipid film is then re-suspended in 10 liters of sterile 0.9% sodium chloride solution with phosphate buffer (pH 7.0-7.4) containing 0.1 mg of purified botulinum type A neurotoxin complex (95-98% purity). After the lipid film is successfully re-suspended from the flask walls, the resulting emulsion is thoroughly mixed for 30 minutes until homogeneous emulsion is produced. Such emulsion is then transferred into a homogenizing reactor and the emulsion is homogenized at a pressure of 60 MPa and a temperature of 30-35 degrees C. The homogenization process is controlled by monitoring optical density values in the vial at a wavelength of 540 nm with a light path thickness of 3 mm. When an optical density of 0.1-0.12 is achieved, 5 grams of lactose is added to the emulsion. The resulting emulsion is then sequentially filtered (for example, in a Millipore, Inc. filtering device), initially through a 0.65 micron filter, then through a 0.45 micron filter, and finally through a 0.22 micron filter. The resulting sterile emulsion is then distributed into vials or ampoules, each containing 0.1 ml of sterile emulsion. The vials or ampoules are deep frozen at a temperature of −70 degrees C. for 48 hours, followed by lyophilization (deep-freeze drying). After lyophilization, the vials are hermetically sealed with an atmosphere of inert gas introduced over the lypophilized emulsion in the vial.

### Example 7

Initially, a flask is filled with a solution of phosphatidylcholine in ethanol containing 0.1 gram of lipid. The solution is subjected to evaporation in a rotating evaporator at a temperature of 30-35 degrees C. until a lipid film is formed. After the evaporation process is completed, an inert gas is passed through the flask of solution for 5 minutes. The lipid film is then re-suspended in 10 liters of sterile 0.9% sodium chloride solution with phosphate buffer (pH 7.0-7.4) containing 0.1 mg of purified botulinum type A neurotoxin complex (95-98% purity). After the lipid film is successfully re-suspended from the flask walls, the resulting emulsion is thoroughly mixed for 30 minutes until homogeneous emulsion is produced. Such emulsion is then transferred into a homogenizing reactor and the emulsion is homogenized at a pressure of 60 MPa and a temperature of 30-35 degrees C. The homogenization process is controlled by monitoring optical density values in the vial at a wavelength of 540 nm with a light path thickness of 3 mm. The resulting emulsion is then sequentially filtered (for example, in a Millipore, Inc. filtering device), initially through a 0.65 micron filter, then through a 0.45 micron filter, and finally through a 0.22 micron filter. The resulting sterile emulsion is then distributed into vials or ampoules, each containing 0.1 ml of sterile emulsion. The vials or ampoules are deep frozen at a temperature of −70 degrees C. for 48 hours, followed by lyophilization (deep-freeze drying). After lyophilization, the vials are hermetically sealed with an atmosphere of inert gas introduced over the lypophilized emulsion in the vial.

### Example 8

Initially, a flask is filled with a solution of phosphatidylcholine in ethanol containing 0.1 gram of lipid. The solution is subjected to evaporation in a rotating evaporator at a temperature of 30-35 degrees C. until a lipid film is formed. After the evaporation process is completed, an inert gas is passed through the flask of solution for 5 minutes. The lipid film is then re-suspended in 10 liters of sterile 0.9% sodium chloride solution with phosphate buffer (pH 7.0-7.4) containing 1 mg of purified botulinum type A neurotoxin complex (95-98% purity). After the lipid film is successfully re-suspended from the flask walls, the resulting emulsion is thoroughly mixed for 30 minutes until homogeneous emulsion is produced. Such emulsion is then transferred into a homogenizing reactor and the emulsion is homogenized at a pressure of 60 MPa and a temperature of 30-35 degrees C. The homogenization process is controlled by monitoring optical density values in the vial at a wavelength of 540 nm with a light path thickness of 3 mm. The resulting emulsion is then sequentially filtered (for example, in a Millipore, Inc. filtering device), initially through a 0.65 micron filter, then through a 0.45 micron filter, and finally through a 0.22 micron filter. The resulting sterile emulsion is then distributed into vials or ampoules, each containing 0.1 ml of sterile emulsion. The vials or ampoules are deep frozen at a temperature of −70 degrees C. for 48 hours, followed by lyophilization (deep-freeze drying). After lyophilization, the vials are hermetically sealed with an atmosphere of inert gas introduced over the lypophilized emulsion in the vial.

What is claimed is:

1. A pharmaceutical preparation comprising:
(a) botulinum neurotoxin;
(b) lactose; and
(c) phosphatidylcholine effectively made into a liposome which incorporates said botulinum neurotoxin.

2. The pharmaceutical preparation of claim 1 wherein said botulinum neurotoxin is 95-99% pure botulinum neurotoxin complex.

3. The pharmaceutical preparation of claim 1 wherein said botulinum neurotoxin is 95-99% pure purified botulinum neurotoxin.

4. The pharmaceutical preparation of claim 1 wherein said botulinum neurotoxin is 95-99% pure botulinum neurotoxin.

5. The pharmaceutical preparation of claim 2 wherein said botulinum neurotoxin complex is selected from the group consisting of botulinum neurotoxin complex types A, B, C1, C2, D, E, F and G.

6. The pharmaceutical preparation of claim 3 wherein said purified botulinum neurotoxin is selected from the group consisting of botulinum neurotoxin complex types A, B, C1, C2, D, E, F and G.

7. The pharmaceutical preparation of claim 4 wherein said botulinum neurotoxin is selected from the group consisting of botulinum neurotoxin types A, B, C1, C2, D, E, F and G.

8. The pharmaceutical preparation of claim 1 wherein said preparation is lyophilized.

9. The pharmaceutical preparation of claim 8 wherein said preparation is stable such that it has substantially no loss of neurotoxin potency for a period of one year when stored at −5 to 37 degrees C.

10. The pharmaceutical preparation of claim 8 wherein said preparation is stable such that it has substantially no loss of neurotoxin potency for a period of four years when stored at −5 to 37 degrees C.

11. The pharmaceutical preparation of claim 8 wherein upon reconstitution, said preparation retains at least 75% of its neurotoxin potency.

12. The pharmaceutical preparation of claim 8 wherein upon reconstitution, said preparation retains at least 75% of its neurotoxin potency.

13. The pharmaceutical preparation of claim 8 wherein upon reconstitution, said preparation retains at least 90% of its neurotoxin potency.

14. A method of making a pharmaceutical preparation, the method comprising:
(a) providing a solution of phosphatidylcholine in ethanol;
(b) subjecting said solution of phosphatidylcholine in ethanol to evaporation until a lipid film is formed;
(c) passing an inert gas through the lipid film;
(d) re-suspending said lipid film in a sterile chloride solution with phosphate buffer contain botulinum neurotoxin creating an emulsion;
(e) homogenizing said emulsion; and
(f) sequentially filtering the homogenized emulsion.

15. The method of claim 14 further comprising the steps of:
(g) distributing the homogenized and filter emulsion into containers;
(h) freezing said emulsion in said containers; and
(i) lyophilizing said emulsion.

16. The method of claim 15 further comprising the step of hermetically sealing said containers having said lyophilized emulsion with an atmosphere of inert gas introduced over the lypophilized emulsion in the container.

17. A method of treating a mammal to achieve a therapeutic effect, the method comprising the step of administering the pharmaceutical preparation of claim 1 to said mammal.

18. A method of treating a mammal to achieve a therapeutic effect, the method comprising:
(a) reconstituting the pharmaceutical preparation of claim 8; and
(b) administering said reconstituted pharmaceutical preparation to said mammal.

19. A pharmaceutical preparation consisting essentially of:
(a) botulinum neurotoxin;
(b) lactose; and
(c) phosphatidylcholine effectively made into a liposome which incorporates said botulinum neurotoxin.

20. The pharmaceutical preparation of claim 19 wherein said botulinum neurotoxin is 95-99% pure botulinum neurotoxin complex.

21. The pharmaceutical preparation of claim 19 wherein said botulinum neurotoxin is 95-99% pure purified botulinum neurotoxin.

22. The pharmaceutical preparation of claim 19 wherein said botulinum neurotoxin is 95-99% pure botulinum neurotoxin.

23. The pharmaceutical preparation of claim 20 wherein said botulinum neurotoxin complex is selected from the group consisting of botulinum neurotoxin complex types A, B, C1, C2, D, E, F and G.

24. The pharmaceutical preparation of claim 21 wherein said purified botulinum neurotoxin is selected from the group consisting of botulinum neurotoxin complex types A, B, C1, C2, D, E, F and G.

25. The pharmaceutical preparation of claim 22 wherein said botulinum neurotoxin is selected from the group consisting of botulinum neurotoxin types A, B, C1, C2, D, E, F and G
